(19) United States
(12) Patent Application Publication
(10) Pub. No.: US 2006/0088480 A1
(43) Pub. Date: Apr. 27, 2006

(54) Oral composition for stabilization, (re)calcification and (re)mineralization of tooth enamel and dentine

(76) Inventor: Robert Basic, Zagreb (HR)

Correspondence Address: KATTEN MUCHIN ROSENMAN LLP, 575 MADISON AVENUE, NEW YORK, NY, 10022-2585, US

(21) Appl. No.: 11/252,353

(22) Filed: Oct. 17, 2005

Related U.S. Application Data
(63) Continuation of application No. PCT/HR04/00010, filed on Apr. 15, 2004.

(30) Foreign Application Priority Data
Apr. 17, 2003 (HR) P20030304A

(51) Int. Cl. A61K 9/68; A61K 8/96

(52) U.S. Cl. 424/48; 424/50

(57) ABSTRACT

An oral composition for the stabilization, (re)calcification and (re)mineralization of dental enamel and dentin to create an efficient protection from tooth decay is based on the use of calcium form of zeolite, phosphate salts soluble in water and matrix proteins of teeth and the adjustment of pH in the mouth cavity to the required value while at the same time incorporating the calcium ions from the calcium form of zeolite into the dental enamel and dentine in the presence of matrix proteins of the teeth. The composition substitutes the calcium and phosphate ions in the structure of hydroxyapatite, stabilizes the crystal structure of calcium hydroxyapatite in tooth enamel and dentin, i.e. in the tooth, along the entire depth of the tooth.

This application is a continuation of International Application PCT/HR2004/000010 filed Apr. 15, 2004, now pending, and claims priority from HR P20030304A, filed Apr. 17, 2003, which is incorporated herein by reference.

### FIELD OF THE INVENTION

This invention refers to finding out an oral composition for stabilization, (re)calcification and (re)mineralization of tooth enamel and dentin, and thus protection of teeth against tooth caries. The method is based on the use of calcium form of zeolite, water-soluble phosphate salts and matrix proteins of tooth for adjustment of pH in mouth cavity to the required value and simultaneous building of calcium ions from zeolite and phosphate ions from solution into tooth enamel and dentin in the presence of tooth matrix proteins, which, by the entire effect, substitutes the calcium and phosphate ions in the structure of hydroxyapatite and stabilizes the crystal structure of calcium hydroxyapatite in tooth enamel and dentin through their entire depths.

### BACKGROUND

Tooth, similarly to other mineralized tissues, is liable to chemical and physical damages on the places “impoverished” by calcium and “enriched” by carbonates. Since the mineral part of tooth consists of sparingly soluble mineral materials, a main reason of the chemical damage of tooth is dissolution of tooth enamel in acidic environment saturated with the components of mineral materials. Impurities, such as sodium, potassium, magnesium, lead, strontium, barium and particularly carbonate ions cause damage of hydroxyapatite crystals and increase of their solubility. Damage of tooth enamel caused by demineralization (dissolution) is accelerated by the action of different endogenic and egsogenic factors such as pregnancy, old age, infancy, osteoporosis, progressive disease of gums and gingivitis; all these factors cause dental lesions.

From the above mentioned reasons, the technical problem for which solution the patent protection is asked, is discovering the effective oral composition for simultaneous stabilization (i.e., decrease of demineralization), (re)calcification and (re)mineralization of tooth enamel and dentin, and in this way, an efficient protection of teeth against caries.

The mechanism of dental caries formation is essentially straightforward; plaque on the surface of tooth consists of bacteria which produces acids as a byproduct of its metabolism. Any fermentable carbohydrate (such as glucose, sucrose, fructose or cooked starch) can be metabolized by the acidogenic bacteria and create the aforementioned organic acids as byproducts. The formed acids diffuse through the plaque into the porous subsurface parts of enamel and dentin. The hydrogen ions formed by dissociation of the mentioned organic acids dissolve the mineral part of enamel and dentin (demineralization). Since the dissolution of mineral part of tooth is favored in acidic environment, process of demineralization is mostly promoted by strong, stable acids: to certain extent, these are found in acid foods, such as tomatoes or oranges. The process of demineralization continues each time when carbohydrate is taken into the mouth. If this process is not halted (by decrease of acidity in the mouth cavity), caries can be developed.

Abundant, well documented investigations carried out through many years, undoubtedly showed a positive effect of fluoride on stabilization of tooth enamel and prevention of tooth caries (J. M. ten Cate and C. van Loveren, Cariology 43 (1999) 713.). Such positive effect of fluoride in the prevention of tooth caries can be simply explained by three fundamental mechanisms (J. D. B. Featherstone, Comm. Dent. Oral Epidemiol. 27 (1999) 31):

(1) Exchange of OH⁻ ions in hydroxyapatite (Ca₅(PO₄)₃OH) by F⁻ ions, i.e.,
Ca₅(PO₄)₃OH+F⁻ [symbol] Ca₅(PO₄)₃F+OH⁻
and by formation of flurapatite (Ca₅(PO₄)₃F) which solubility in acidic environment is about ten times lower than the solubility of hydroxyapatite, and in this way, by stabilization of enamel and dentin by slowing down the process of demineralization (J. D. B. Featherstone, R. Glena, M. Shariati and CP. Shields, J. Dent. Res. 69 (1990) 20.; J. M. ten Cate and J. D. B. Featherstone, Crit. Rev. OralBiol. 2 (1991)
283.)
   (2) Enhancing remineralization on the surface by acceleration of the processes of crystallization of hydroxyapatite and fluorapatite.
   (3) Inhibiting the growth of cariogenic bacteria by collecting HF in their cells; investigations have shown that F⁻ ions which exist in neutral and alkaline media cannot pass the walls and membrane of cells, but that HF which exists in acidic medium easily passes the walls and membrane of cells (G. M. Whitford, G. S. Schuster and H. D. Pashley, Infect. Immun. 18 (1977) 680.; C. Van Louveren, J. Dent. Res. 69 (1990) 676.; I. R. Hamilton and G. W. H. Bowden in: 0. Fjereskov, J. Ekstrans and B. A. Burt (eds.), Fluoride in Dentistrv, Munksgaard, Copenhagen, 1996, p. 230.). Development of caries increases the acidity of medium causing formation of HF in the presence of F⁻ ions, and the formed HF collects in the cells and, in this way stops their further growth. Due to the mentioned effect of fluorine, fluorine compounds are widely used in dental medicine, which is evidenced in numerous scientific publications (for this purpose see the reviews: J. M. ten Cate and C. van Loveren, Cariology 43 (1999) 713 and J. D. B. Featherstone, Comm. Dent. Oral Epidemiol. 27 (1999) 31) and patents (for eg. K. Brigham and R. C. Vickerv, U.S. Pat. No. 3,647,488, 1972; Tomlinson and E. J. Duff, U.S. Pat. No. 4,048,300; M. C. S. Gaffar and A. Gaffar, U.S. Pat. No. 4,177,258; M. C. S. Gaffar and A. Gaffar, U.S. Pat. No. 4,183,915; J. Weststrate and E. M. Staal, U.S. Pat. No. 4,460,565; W. Schmidt, R. Purrmann, P. Jochum and H. J. Huebner, U.S. Pat. No. 4,472,836; J. J. Paran, Jr. and N. Y. Sakkab, U.S. Pat. No. 4,515,772; N. Usen and A. E. Winston, U.S. Pat. No. 5,605,675; A. E. Winston and N. Usen, U.S. Pat. No. 5,817,296; A. E. Winston and N. Usen, U.S. Pat. No. 5,858,333; N. Usen and A. E. Winston, U.S. Pat. No. 5,895,641; R.-R. Miethke and H. Newesely, German Patent DE 3,404,827; T. Reetz, S. Zimmer and W. Krahl, German Patent DE 19,735,929; J. W. Stansburry, J. M. Antonucci and K. M. Choi, U.S. Pat. No. 6,184,339; F. Rueggeberg, G. Whitford and D. Mettenburg, US Patent Appl. Publ. 2002028856, 2002).

However, due to knowledge that remineralization by fluorine is effective only in the presence of calcium and phosphate ions (A. Papas, D. Russell, M. Singh, K. Stack, R. Kent, C. Triol, et al, Gerodontol 16 (2000) 2.) and the growing concerns connected with negative effects of fluorine on the human health, there is a need to develop new approaches for preventing of caries and processes of remineralization (M. S. Tung and F. C. Eichmiller, J. Clin. Dent. 10(1999)1.).

N. Randol (U.S. Pat. No. 3,934,267: Method for remineralizing and immunizing tooth enamel for the prevention and control of tooth decay and dental caries) developed a method based on the treatment of enamel with acid with an intention to remove the positively charged calcium, which causes the formation a porous sponge-like negatively charged surface. Surface of enamel prepared in the describe way was treated with the solution of positively charged heavy metal ions which, by electrostatic forces, depose on the negatively charged surface of enamel. In addition, tooth enamel containing heavy metals on the surface was additionally treated with sulfur compounds in order to form heavy metal sulfides which are resistant on the acids formed during development of caries, and in this way protect teeth against decay.

K. Tomlinson and E. J. Duuf (U.S. Pat. No. 4,048,300: Dental preparation containing materials having calcium and phosphate components) described a preparation (cream) for remineralization of tooth enamel containing fluorapatite, fluorhydroxyapatite and hydroxyapatite, and the materials which contain monofluorphosphate, and carbonate or two-valent ions such as ZnF₄²⁻.

D. N. DiGiulio and R. J. Grabenstetter (U.S. Pat. No. 4,080,440: Method for remineralizing tooth enamel): developed a method based on the treatment of tooth enamel with metastable water solution of calcium (0.005%-5%) and phosphate (0.005%-5%) ions with the molar ratio Ca:P between 0.01 and 100 and pH 2.5-4. The solution can be used 5 min after preparation and duration of application (keeping in the mouth cavity) is between 10 seconds and 3 minutes, i.e. during the time interval in the solution is metastable. Remineralization occurs by incorporation of calcium and phosphate ions from solution in the deminaralised surfaces of teeth.

R. J. Grabenstetter and J. A. Gray (U.S. Pat. No. 4,083,955: Processes and compositions for remineralization of dental enamel) developed a method of remineralization in two stages. In the first stage, mouth cavity is treated with 0.005-10% water solution of soluble calcium ions or 0.005-10% water solution of soluble phosphate ions. During the treatment (10-30 seconds) calcium or phosphate ions build into surface and subsurface parts of tooth enamel. Insofar as the mouth cavity was treated by calcium ions in the first stage, the mouth cavity was treated with phosphate ions in the second stage for the same time (10-30 seconds), and vice versa. During the second stage of the treatment, phosphate ions from solution react with the calcium ions previously built into enamel, and calcium ions from solution react with the phosphate ions previously built into enamel, respectively, forming hydroxyapateite in both the cases.

M. C. S. Gaffar and A. Gaffar (U.S. Pat. No. 4,177,258: Dentifrice for dental remineralization) developed a preparation for nursing of teeth containing a source of calcium ions (water solution containing 50 ppm of calcium ions), a source of phosphate ions (water solution containing 50 ppm of phosphate ions; Ca:P=0.01-100), a source of fluoride ions, a gel for stabilization of calcium and phosphate ions and a compound for prevention of nucleation (ethylene-diamine-tetramethylenphosphonic acid or its water soluble salts). pH of preparation is 5-9, desirable 6.8-7.5 (physiological conditions).

W. M. Jarvis and K. Y. Kim (U.S. Pat. No. 4,244,931: Dicalcium phosphate dehydrate with improved stability) developed a preparation for polishing of teeth containing Dicalcium phosphate, a sufficient amount of trimagnesiumphosphate and/or pyrophosphate which prevent a spontaneous decomposition of Dicalcium phosphate dehydrate.

H. Raaf, H. Harth and H. R. Wagner (U.S. Pat. No. 4,397,837: Process and composition for the remineralization and prevention of animal teeth including humans) developed a preparation for remineralization and prevention of demineralization of dental enamel, containing two phases; one phase contains water solution of calcium salts (50-35000 ppm and 0.005 wt. %-3.5 wt. %, respectively), and another phase contains water solution of phosphate (50-40000 ppm and 0.004 wt. %-4 wt. %, respectively) and water solution of fluoride (0.01 wt. %-5 wt. %). The preparation can also contain polishing agent, densication agent and conservans.

A. G. Kolesnik, G. I. Kadnikova, L. V. Morozova and L. M. Boginskaya (U.S. Pat. No. 4,419,341: Drug for treatment of dental caries) described a procedure to prepare preparation for prevention of caries. A solution prepared by dissolution of mineral component and water soluble proteins from bone tissue by diluted mineral acid, was diluted by water, and the stabilizer (lemon acid or lemon acid salt) was added in such diluted solution. The obtained solution was neutralized and evaporated and then mixed with pharmaceutical dilutant in the ratio 1:23.5-1:24.5.

J. Weststrate and E. M. Staal (U.S. Pat. No. 4,460,565: Anticariogenic remineralizing dentrifice) developed a preparation for remineralization containing 1000-15000 ppm of F ions (depending on application) applied in the form of alkaline fluorides, earth-alkaline fluorides, ammonium fluoride and alkaline fluorophosphates, 0.1-5 wt. % soluble cyclic alkaline phosphates, 0.05-5% potassium citrate and/or calcium tartarate and soluble linear phosphates so that atomic ratio Ca:P is about 1.66:1.

J. J. Paran Jr. and N. Y. Sakkaab (U.S. Pat. No. 4,515,772: Oral compositions) developed a preparation for protection of teeth in the for of tooth-paste containing 10-70% abrasive (metaphosphates, aluminum trioxide, polymerized resins and amorphous silica), 50-3500 ppm F ions and at least 1.5% P₂O₇⁴⁻ ions (added in the form of dialkali metal and tetraalkali metal pyrophosphates) and water. The preparation contains maximum 4% K₄P₂O₇, and pH of the preparation is between 6 and 10.

M. A. Rudy and V. F. Lisanti (U.S. Pat. No. 4,606,912: Method for making a clear, stable aqueous mouthwash solution and the solution made by that method for the enhancement of cells of the oral cavity and the remineralization of teeth) described the preparation of the mouthwash solution effective in the prevention of development of caries and reduction of unpleasant odor. The solution contains calcium chelates in which minimally 50% of calcium ions is chelated. The solution is weakly alkaline.

F.-J. Dany, H. Klassen, H. Prell and G. Kalteyer (U.S. Pat. No. 4,931,272: Tooth pastes, cleaning agent for tooth pastes based on dicalcium phosphate-dihydrate, and process for making such cleaning agent) described a procedure for preparation of the toothpaste containing dicalcium phosphate-dihydrate as main active component. The mentioned toothpaste contains more than 60% of water per 100 g of active component.

M. J. Greenberg (U.S. Pat. No. 5,378,131: Chewing gum with dental health benefit employing calcium glycerophosphate) described preparation of chewing gum (without fluorides) that prevents the development of dental caries and enhances the dental hygiene especially after meals containing fermentable carbohydrates. The chewing gum contains minimally 0.5 wt. % calcium glycerophosphate.

A. E. Winston and N. Usen (U.S. Pat. No. 5,603,922: Processes and compositions for the remineralization of teeth) described the preparation for remineralization of teeth which contains two components: On of the components contains 0.05-15% one or more water soluble calcium salts and 0.001-2% one or more water soluble divalent metals different than calcium. Another component contains 0.05-15% one or more water soluble phosphate salts. After mixing together both the components, a stable solution having pH between 4 and 7 is formed. During application (contact with teeth) remineralization occurs by diffusion of calcium and phosphate ions through the solution to the surface of teeth, where hydroxyapatite is formed by reaction between calcium and phosphate ions.

A. E. Winston and N. Usen (U.S. Pat. No. 5,614,175: Stable single-part compositions and the use of thereof for remineralization of lesions in teeth) described non-water composition for remineralization of teeth and its application. The composition consists of 0.05-15% one or more water soluble calcium salts, 0.05-15% one or more water soluble phosphate salts, stabilizer and up to 7% compounds for drying and coating. pH of the composition is between 4.5 and 10.

A. E. Winston and N. Usen (U.S. Pat. No. 5,645,853: Chewing gum compositions and the use of thereof for remineralization of lesions in teeth): described the chewing gum containing 0.01-15% % one or more water soluble calcium salts, 0.01-15% one or more water soluble phosphate salts, 10-95% gum base and a layer for encapsulation. During chewing, both calcium and phosphate ions are released from the gum and together with saliva form a mixed solution of calcium and phosphate ions having pH between 4 and 7. Phosphate and calcium ions from saliva deposit on the tooth surface where react and induce remineralization by crystallization of calcium phosphate (hydroxyapatite).

L. C. Chow and S. Takagi (U.S. Pat. No. 5,695,729: Calcium phosphate hydroxyapatite precursor and methods for making and using the same) describes a procedure for preparation of a composition of calcium phosphate usable in orthopedic and dental cements and remineralizers. Composition consists of tetracalcium phosphate prepared by a mixture of calcium and phosphorus in the ratio less than 1:2.

A. E. Winston and N. Usen (U.S. Pat. No. 5,817,296: Processes and compositions for the remineralization of teeth) developed procedure for preparation of a stable non-water dry composition which form a water solution usable for remineralization of teeth after dissolution in water. The composition is prepared by dry mixing of one or more water soluble calcium salts (1-80%), one or more water soluble non-toxic salts of divalent metals different from calcium (0.1-20%), one or more water soluble phosphate salts, flavor (0.1-20%), sweetener (0.1-30%), one or more fluoride salts (0-10%) and surface active substances (about 5%). pH of water solution of such prepared composition is between 4 and 7.

L. C. Chow, S. Takagi and G. L. Vogel (U.S. Pat. No. 5,833,954: Anti-carious chewing gums, candies, gels, toothpastes and dentifrices) described two-component preparation for remineralization subsurface lesions and/or exposed dental tabulus in tooth. Cationic component contains one or more water soluble calcium salts, one or more water soluble non-toxic salts of divalent metals different from calcium and pharmaceutically acceptable carrier. Anionic component contains one or more water soluble calcium salts, one or more water soluble fluoride salts and pharmaceutically acceptable carrier. If carrier of cationic component is water one, then carrier of anionic component is non-water (hydrophobic) one, and if carrier of anionic component is water one, then carrier of cationic component is non-water, respectively. Mixing of both the components with water or saliva causes simultaneous releasing of calcium and phosphate ions and their mutual reaction on the tooth surface. If the contact of the remineralizing solution and tooth is long enough, calcium and phosphate ions diffuse through the surface of tooth which enables remineralization of lesions and tabulus.

N. Usen and A. E. Winston (U.S. Pat. No. 5,895,641: Process and composition for remineralization and prevention of demineralization of dental enamel) described the method of remineralization of lesions and tabulus in the subsurface layer of tooth, which consists of (1) Preparation of cationic component containing 0.05-15% one or more water soluble calcium salts (calcium chloride or calcium nitrate. (2) Preparation of anionic component containing 0.05-15% one or more water soluble and 0.01-5% water soluble fluoride salts. (3) Water solution having pH between 4.5 and 10 was formed after mixing together anionic and cationic components; the solution contains free calcium ions released from calcium salts, free phosphate ions released from phosphate salts and free fluoride ions released from fluoride salts. (4) Application of the solution immediately after preparation causes reaction of calcium, phosphate and fluoride ions on the surface of tooth. If the contact of the remineralizing solution and tooth is long enough, calcium and phosphate ions diffuse through the surface of tooth which enables remineralization of lesions and tabulus.

A. E. Winston and N. Usen (U.S. Pat. No. 6,036,944: Process for remineralization of teeth) described a method of remineralization of subsurface lesions and dental tabulus by preparation of components containing one or more water soluble calcium salts, one or more water soluble non-toxic salts of divalent metals different from calcium, one or more water soluble phosphate salts and mixing together the mentioned components so that the formed carbonateless solution has pH between 4.5 and 7. If the contact of the remineralizing solution and tooth is long enough, calcium and phosphate ions diffuse through the surface of tooth which enables remineralization of lesions and tabulus.

Some of new approaches in remineralization use the properties of amorphous calcium phosphates (ACP) (M. S. Tung, U.S. Pat. No. 5,037,639; M. S. Tung, T. O'Farrell and D. W. Liu, J. Dent. Res. 72 (1993) 320.). Among different forms of calcium phosphates, ACP exhibits the maximum rate of formation, but also the maximum solubility, whereas ACP rapidly hydrolyzes into crystalline apatite ((E. D. Eanes in: Z. Amjad (Ed.), Calcium Phosphates in Biological and Industrial Systems, Kluwer Academic Pub., Boston, 1998, p. 21.). High concentrations of calcium and phosphate ions from their primary sources (soluble salts) rapidly precipitate ACP during their application as preparations for rinsing and nursing of teeth ((M. S. Tung, M. Markovic and T. J. O'Farrell, J. Dent. Res. 73 (1994) 1903.; M. S. Tung, J. Dent. Res. 75 (1996) 56.).

J. D. Termine, R. D. Peckauskas and A. S. Posner (Arch. Biochim. Biophys. 140 (1970) 318) established that when ACP is stabilized with pyrophosphate (P₂O₇⁴⁻), supersaturated solution keeps stable for a longer time, i.e., that does not occur a spontaneous crystallization of the crystal forms of calcium phosphate.

Based on this findings, D. Skrtic, E. D. Eanes and J. M. Antonucci (in: C. G. Gebelein, C. E. Carraher, Jr. (Eds), Industrial Biotechnological Polymers, Technomic, Lancaster, Pa., 1995, p. 393) made discs formed of ACP stabilized with pyrophosphate incorporated in metaacrilic resins). When such discs are immersed in buffered salted solution, they release calcium and phosphate ions in the concentrations sufficient to form stable solution supersaturated with respect to hydroxyapatite.

Soluble ACP may be applied as an additive in the chewing gum; calcium, phosphate, fluoride, bicarbonate and hydroxyl ions needed for remineralization and regulation of pH in the dental cavity are released during chewing (M. S. Tung and F. C. Eichmiller, J. Clin. Dent. 10 (1991) 1.).

The most known preparation for remineralization of teeth based on ACP is sugarless chewing gum Recaldent™ (Patent) in which the source of calcium and phosphate ions is ACP stabilized by caseine (part of protein from cow milk). Recaldent™ is developed and patented at School of Dental Science (The University of Melbourne, Australia) and exclusively licensed by the company Bonlac Foods.

R. S. Schreiber and J. R. Principe (U.S. Pat. No. 4,187,287: Warm Two Tone Flavored Dentifrice) described application of dehydrated forms of zeolite 3A, 4A and 5A as well as zeolite X as the components for increase of temperature of oral compositions, and hence enhancement of effect of flavor components. At the same time, abrasive and polishing effects of zeolite may reduce the amounts of other abrasive and polishing agents. Recaldent™

However, while the abrasive and polishing effects of zeolite are doubtless, the effect of “warming up” is time-limited because of reversible character of adsorption and desorption of zeolitic water. Or in the other words, thermal effect can be effective just in the moment of mixing of zeolite with other components of the oral composition, but this effect completely disappears after certain time passed between the preparation and application of the oral composition.

L. Dent, E. P. Hertzenberg and H. S. Sherry (U.S. Pat. No. 4,349,533: Toothpaste containing pH-adjusted zeolite) described the method of adjusting pH value of oral compositions in the range pH=5.5 to pH=6 using of zeolite NaHA, CaHA, MgHA, NaHX, CaHX, ZnHX, MgHX and their mixtures obtained by ions exchange and acid treatment (modification) of sodium forms of the mentioned zeolite.

Besides pH achieved by mentioned zeolite is too low for effective prevention of demineralization processes, zeolite modified by acid treatment are unstable ant tend to transform into amorphous aluminosilicates (especially zeolite A) both during acid treatment and during the time passed from preparation of oral composition to its application.

### FIGURES

FIG. 1 is a chart of the dependence of pH of the oral composition about the logarithm of concentration log Cp, PO₄³⁻(O), HPO₄²⁻(Δ) and H2PO₄⁻ ions.

### DETAILED DESCRIPTION OF THE INVENTION

Given methods and respective preparations for dental hygiene use in water soluble calcium salts as a source of calcium ions and in the water soluble phosphate salts as a source of phosphatic ions in the process of remineralization of teeth. However, implementing water soluble and/or partially soluble calcium and phosphate salts as a source of calcium and phosphate ions in the agents for the remineralization, induces difficulties connected with the control of concentration of calcium and phosphate ions; if the concentrations of the mentioned ions are too low, one cannot reach the required level of remineralization, on the other hand, too high concentrations of the mentioned ions can cause crystallization of apatites with defective crystalline structure and/or unwanted crystal conglomerates on the surface of teeth. Specific problem is a permanent change of the concentration of calcium and phosphate ions in the solution during the process of remineralization. Furthermore, in water soluble and/or partially soluble calcium and phosphate salts are the resource of various negative anions (chlorides, nitrate bicarbonates etc.) which may have negative impact on the crystallization of hydroxyapatite and on the stability (solubility) of dental enamel. Furthermore, due to relatively low concentrations of phosphates and its further reduction during the remineralization, appears the reduction of pH (the increase of acidity) in the dental cavity which may cause slowing down of the process of remineralization or even an increase in velocity of demineralization. Since the control of acidity (pH) in the dental cavity is one of the most significant factors for the control of stability of mineral portion of enamel and dentine, and in this way controls the development of dental caries (M. E. Jensen, Cariology 43 (1999) 615.), in the mentioned methods and appropriate preparations, pH is controlled by <<traditional>> methods, i.e. additional bicarbonate and/or urea. Though the bicarbonate present in the preparations for the dental hygiene reduces the sourness and in this way increases the stability of mineral part of the teeth, the presence of carbonates can have negative impact on the stability of dental enamel and dentine. Namely, OH⁻ ions in the hydroxyapatite can be replaced with carbonate ions from the solution, and in this way create carbonated apatites which are, in sour environment, greatly more soluble than hydroxyapatite. Urea, although neutral substance, in the sour area caused by the activities of cariogenic bacteria, hydrolyses on ammonium and carbon dioxide, where the created ammonium neutralizes the acid and in this way reduces the sourness in the mouth cavity. However, the new studies that compared the use of commercial chewing gums who did not contain urea and chewing gums who did contain urea, have proven the presence of urea has no significant effect of pH in the dental cavity (D. Birkhed, J. Dent Res. 68 (Special Issue) (1989): Abstract 1027; T. Imfeld, Telemetric Evaluation discourse the plaque-pH Neutralize Potential of two Chewing Gums Provided by Fertin A/S., Intermural Paper, Dental Institute of Zurich, September 1996.). Furthermore, it is known that ammonium reduces the lifetime of cells and induces the growth of humane ginvival fibroblasts in vivo (K. Helgeland, Scand. J. Res. 89 (1981) 400) Furthermore, ammonium negatively affects the emission of collagen by virtue of cells and in this way can provoke periodontal inflammation and tissue breakdown breakdown (K. Helgeland, Scand. J. Dent. Res. 92 (1984) 419.; K. Helgeland, Scand. J. Dent. Res. 93 (1981) 39.). Finally, carbon dioxide created as a product hydrolysis of urea can create carbonate ions which in the hydroxyapatite can be replaced by OH⁻ ions and in this way create carbonated apatites who are in sour environment

much more soluble than hydroxyapatites. Hence follows that the utilization of urea is not only questionable relating to the effect of neutralization of acidity, but can cause even the health problems.

Considering the above mentioned difficulties in controlling the pH (i.e. the use of bicarbonate, urea and acidically modified zeolite for that purpose) and concentrations of calcium and phosphatic ions in the so far developed preparations for remineralization of teeth, the present invention represents the new oral composition for stabilization, recalcifikation and remineralization of dental enamel and dentine founded on the controlled freeing of calcium ions from the calcium form of zeolite in the presence of water soluble phosphate salts with or without dental matrix proteins. Since the calcium ions are bound in the micro crystalline particles of inorganic carriers (zeolite), they are not active until “free” from the crystalline grid of zeolite. Hence follows, that although the concentrations of calcium and phosphate ions in the oral composition are favorable (or more than favorable) for total remineralization, the speed of remineralization is controlled by the rates of release of calcium ions from the microcrystall zeolite during the ionic substitution of calcium ions from the zeolite with other ions from the solution. Calcium ions, freed from the microcrystall of zeolite are implanted together with equivalent quantity of phosphate ions and dental matrix proteins from the solution in the dental enamel and dentin, and in this way they remineralize them. In the design of the innovation with dental matrix proteins, the same improve the above mentioned processes in the following manner: dental matrix protein occurs as the precursor of maturation, from “unripe” calcium hydroxyapatite into “ripe” calcium hydroxyapatite, whose solubility is less than that of the “unripe” calcium hydroxyapatite. Specified components of calcium hydroxyapatite; calcium ions, phosphate ions and dental matrix protein fully construct the dental enamel in the process of self-organized structures (restitutio ad integrum).

Oral composition, according to the invention, can be used in the form of dental paste, chewing gum, gel, mouth rinsing water, bonbon and other preparations which are kept in the mouth cavity. All mentioned applications of oral composition of innovation additionally can contain some extraction and/or the oil of the medicinal herb milfoil as a mild anti-inflammatory agent, in the quantities from about 0.01 to about 0,025 wt. %.

Although the essential role of the calcium form of zeolite in the oral composition for the stabilization, recalcification and remineralization of dental enamel (OKSRCD) based on this invention is the <<provision>> of the system with active calcium ions, calcium build zeolite has also another extremely important role, and it is the control and maintenance of optimal pH for the stabilization and remineralization of dental enamel and dentine.

On the other hand, relating to broadly used soluble phosphate salts for regulation of pH in the biological systems (J. Gabelberger, W. Liebl and K. H. Schleifer, Appl. Microbiol. Biotechnol. 40 (1993) 44.; K. Uchida and S. Kawasaki, J. Biol. Chem. 269 (1994) 2405.; Y. Kawata and K. Hamaguchi, Protein Sci. 4 (1995) 416.; F. Ruizteran and J. D. Owens, Lett. Appl. Microbiol. 22 (1996) 30.; K. Brogden and C. Clarke, Infect. Immun. 65 (1997) 957.; H. Chen and M. R. Juchau, Drug Metabolism Disposition 26 (1998) 222.; P. E. Jorgensen, L. Eskildsen and E. Nexo, Scand. J. Clin. Lab. Invest. 59 (1999) 191.; O. Castejon and P. Sims, Biocell. 23 (2000) 187.; Y. Bai and Z. L. Nikolov, Biotechnol Prog. 17 (2001) 168.; C. K. Chang, V. Simplaceanu and C. Ho, Biochemistry 41 (2002) 5644.) including the methods and preparations used in the dental hygiene (K. Tomlinson and E J. Duuf, U.S. Pat. No. 4,048,300; D. N. DiGiulio and RJ. Grabenstetter, U.S. Pat. No. 4,080,440; RJ. Grabenstetter and J. A. Gray, U.S. Pat. No. 4,083,955; M. C. S. Gaffar and A. Gaffar, U.S. Pat. No. 4,177,258; H. Raaf, H. Harth and H. R. Wagner, U.S. Pat. No. 4,397,837; A. E. Winston and N. Usen, U.S. Pat. No. 5,603,922; A. E. Winston and N. Usen, U.S. Pat. No. 5,614,175; A. E. Winston and N. Usen, U.S. Pat. No. 5,645,853; A. E. Winston and N. Usen, U.S. Pat. No. 5,817,296; L. C. Chow, S. Takagi and G. L. Vogel, U.S. Pat. No. 5,833,954; N. Usen and A. E. Winston, U.S. Pat. No. 5,895,641; A. E. Winston and N. Usen, U.S. Pat. No. 6,036,944); soluble salts of various manifested forms (POˆ″, HPOˆ″, H2PO4″) phosphatic can be used for regulation of pH in the mouth cavity, even more because the phosphate ion is one of the essential ingredients of natural apatites-mineral part of dental enamel and dentine.

Considering that the processes of stabilization, demineralization and remineralization of dental enamel largely depends upon the pH of liquid in the dental cavity, and thus also the pH of mentioned oral composition, it is extremely important to know the dependence of pH of mentioned oral composition about its content in relation to the content of calcium form of zeolite and phosphate salts. Concentration of dental matrix proteins in the oral composition of the innovation is too small to have a more significant effect on the pH of the same oral composition. On the pH of oral composition, above all influences the concentration of phosphate ions and the quality and type of Ca form of zeolite. The results of testing the impact of the mentioned components in the oral composition on the pH of the same composition, were shown in the table 1.

The influence of the type of calcium form of zeolite (CaZ), weight percent of calcium form of zeolite (wt. % CaZ), chemical forms of phosphatic ion (the form of phosphate), weight percent of phosphatic ion (wt. % of phosphate) and molecular concentrations of phosphatic ions on the molar ratio Ca/P on the equilibrium of pH amount of oral composition for stabilization, (re)calcification and (re)mineralization of dental enamel and dentine. Weight ratios of matrix proteins are not mentioned in the table, because they have no influence on the pH.

TABLE 1

| | | | | | | | state of |
|---|---|---|---|---|---|---|---|
| | type | weight | the form of | wt. % of | molar concentration of | molar | equilibrium of |
| No. | CaZ | % CaZ | phosphate | phosphate | phosphate mol dm-³ | ratio Ca/P | pH |
| 1. | I | 0.1 | — | 0 | 0 | ∞ | 7.50 |
| 2. | I | 0.1 | PO₄³⁻ | 0.1586 | 0.0167 | 0.16 | 11.70 |
| 3. | I | 0.1 | PO₄³⁻ | 0.01586 | 0.00167 | 1.6 | 9.80 |
| 4. | I | 0.5 | PO₄³⁻ | 0.1586 | 0.0167 | 0.8 | 11.68 |
| 5. | I | 1.0 | — | 0 | 0 | ∞ | 9.07 |
| 6. | I | 1.0 | PO₄³⁻ | 0.1586 | 0.0167 | 1.6 | 11.72 |
| 7. | I | 1.0 | PO₄³⁻ | 0.075 | 0.0079 | 3.38 | 11.31 |
| 8. | I | 1.0 | PO₄³⁻ | 0.0076 | 0.0008 | 33.4 | 8.40 |
| 9. | I | 1.0 | PO₄³⁻ | 0.00715 | 0.00075 | 35.6 | 8.30 |
| 10. | I | 1.0 | PO₄³⁻ | 0.00355 | 0.00037 | 71.4 | 7.96 |
| 11. | I | 1.0 | PO₄³⁻ | 0.00143 | 0.00015 | 178 | 8.15 |
| 12. | I | 5.0 | PO₄³⁻ | 1.586 | 0.167 | 0.8 | 11.72 |
| 13. | I | 10.0 | — | 0 | 0 | ∞ | 9.18 |
| 14. | I | 10.0 | PO₄³⁻ | 1.586 | 0.167 | 1.6 | 11.74 |
| 15. | I | 0.1 | HPO₄²⁻ | 0.1586 | 0.0167 | 0.16 | 8.16 |
| 16. | I | 0.1 | HPO₄²⁻ | 0.01586 | 0.00167 | 1.6 | 8.20 |
| 17. | I | 0.5 | HPO₄²⁻ | 0.1586 | 0.0167 | 0.8 | 8.00 |
| 18. | I | 1.0 | HPO₄²⁻ | 1.586 | 0.167 | 0.16 | 8.05 |
| 19. | I | 1.0 | HPO₄²⁻ | 0.632 | 0.0665 | 0.4 | 8.10 |
| 20. | I | 1.0 | HPO₄²⁻ | 0.316 | 0.0333 | 0.8 | 7.96 |
| 21. | I | 1.0 | HPO₄²⁻ | 0.1586 | 0.0167 | 1.6 | 8.10 |
| 22. | I | 1.0 | HPO₄²⁻ | 0.0534 | 0.00562 | 4.75 | 7.92 |
| 23. | I | 1.0 | HPO₄²⁻ | 0.027 | 0.00284 | 9.4 | 7.85 |
| 24. | I | 1.0 | HPO₄²⁻ | 0.00726 | 0.000764 | 34.95 | 7.81 |
| 25. | I | 1.0 | HPO₄²⁻ | 0.00395 | 0.000416 | 64.18 | 7.80 |
| 26. | I | 1.0 | HPO₄²⁻ | 0.00132 | 0.000139 | 182 | 7.85 |
| 27. | I | 5.0 | HPO₄²⁻ | 1.586 | 0.167 | 0.8 | 8.16 |
| 28. | I | 10.0 | HPO₄²⁻ | 1.586 | 0.167 | 1.6 | 8.15 |
| 29. | I | 0.1 | H₂PO₄⁻ | 0.1586 | 0.0167 | 0.16 | 6.93 |
| 30. | I | 0.1 | H₂PO₄⁻ | 0.01586 | 0.00167 | 1.6 | 7.20 |
| 31. | I | 0.5 | H₂PO₄⁻ | 0.1586 | 0.0167 | 0.8 | 6.82 |
| 32. | I | 1.0 | H₂PO₄⁻ | 0.158 | 0.0167 | 1.6 | 6.78 |
| 33. | I | 1.0 | H₂PO₄⁻ | 0.0071 | 0.00075 | 35.7 | 7.60 |
| 34. | I | 1.0 | H₂PO₄⁻ | 0.00364 | 0.00038 | 69.5 | 7.90 |
| 35. | I | 1.0 | H₂PO₄⁻ | 0.00194 | 0.000204 | 130.5 | 7.68 |
| 36. | I | 5.0 | H₂PO₄⁻ | 1.586 | 0.167 | 0.8 | 6.56 |
| 37. | I | 10.0 | H₂PO₄⁻ | 1.586 | 0.167 | 1.6 | 6.65 |
| 38. | II | 0.1 | — | 0 | 0 | ∞ | 6.10 |
| 39. | II | 0.1 | PO₄³⁻ | 0.001586 | 0.000167 | 12.8 | 8.15 |
| 40. | II | 0.1 | PO₄³⁻ | 0.01586 | 0.00167 | 1.28 | 9.62 |
| 41. | II | 0.1 | PO₄³⁻ | 0.1586 | 0.0167 | 0.128 | 11.76 |
| 42. | II | 1.0 | — | 0 | 0 | ∞ | 6.35 |
| 43. | II | 1.0 | PO₄³⁻ | 0.001586 | 0.000167 | 128 | 8.17 |
| 44. | II | 1.0 | PO₄³⁻ | 0.01586 | 0.00167 | 12.8 | 9.76 |
| 45. | II | 1.0 | PO₄³⁻ | 0.1586 | 0.0167 | 1.28 | 11.78 |
| 46. | II | 10.0 | — | 0 | 0 | ∞ | 7.90 |
| 47. | II | 10.0 | PO₄³⁻ | 0.01586 | 0.00167 | 128 | 9.58 |
| 48. | II | 10.0 | PO₄³⁻ | 0.1586 | 0.0167 | 12.8 | 11.67 |
| 49 | II | 10.0 | PO₄³⁻ | 1.586 | 0.167 | 1.28 | 11.82 |
| 50. | II | 0.1 | HPO₄²− | 0.001586 | 0.000167 | 12.8 | 7.85 |
| 51. | II | 0.1 | HPO₄²− | 0.01586 | 0.00167 | 1.28 | 7.96 |
| 52. | II | 0.1 | HPO₄²− | 0.1586 | 0.0167 | 0.128 | 8.12 |
| 53. | II | 1.0 | HPO₄²− | 0.001586 | 0.000167 | 128 | 7.78 |
| 54. | II | 1.0 | HPO₄²− | 0.01586 | 0.00167 | 12.8 | 7.91 |
| 55. | II | 1.0 | HPO₄²− | 0.1586 | 0.0167 | 1.28 | 8.05 |
| 56. | II | 10.0 | HPO₄²− | 0.01586 | 0.00167 | 128 | 8.00 |
| 57. | II | 10.0 | HPO₄²− | 0.1586 | 0.0167 | 12.8 | 8.06 |
| 58. | II | 10.0 | HPO₄²− | 1.586 | 0.167 | 1.28 | 8.16 |
| 59. | II | 0.1 | H₂PO₄⁻ | 0.001586 | 0.000167 | 12.8 | 7.78 |
| 60. | II | 0.1 | H₂PO₄⁻ | 0.01586 | 0.00167 | 1.28 | 7.44 |
| 61. | II | 0.1 | H₂PO₄⁻ | 0.1586 | 0.0167 | 0.128 | 6.60 |
| 62. | II | 1.0 | H₂PO₄⁻ | 0.001586 | 0.000167 | 128 | 7.80 |
| 63. | II | 1.0 | H₂PO₄⁻ | 0.01586 | 0.00167 | 12.8 | 7.54 |
| 64. | II | 1.0 | H₂PO₄⁻ | 0.1586 | 0.0167 | 1.28 | 6.66 |
| 65. | II | 10.0 | H₂PO₄⁻ | 0.01586 | 0.00167 | 128 | 7.65 |
| 66. | II | 10.0 | H₂PO₄⁻ | 0.1586 | 0.0167 | 12.8 | 6.76 |
| 67. | II | 10.0 | H₂PO₄⁻ | 1.586 | 0.167 | 1.28 | 6.28 |
| 68. | III | 0.1 | — | 0 | 0 | ∞ | 4.88 |
| 69. | III | 0.1 | PO₄³⁻ | 0.001586 | 0.000167 | 9.6 | 8.07 |
| 70. | III | 0.1 | PO₄³⁻ | 0.01586 | 0.00167 | 0.96 | 9.46 |
| 71. | III | 0.1 | PO₄³⁻ | 0.1586 | 0.0167 | 0.096 | 11.56 |
| 72. | III | 1.0 | — | 0 | 0 | ∞ | 5.50 |
| 74. | III | 1.0 | PO₄³⁻ | 0.001586 | 0.000167 | 96 | 8.08 |
| 75. | III | 1.0 | PO₄³⁻ | 0.01586 | 0.00167 | 9.6 | 9.52 |
| 76. | III | 1.0 | PO₄³⁻ | 0.1586 | 0.0167 | 0.96 | 11.63 |
| 77. | III | 10.0 | — | 0 | 0 | ∞ | 6.85 |
| 77. | III | 10.0 | PO₄³⁻ | 0.01586 | 0.00167 | 0.96 | 9.61 |
| 78. | III | 10.0 | PO₄³⁻ | 0.1586 | 0.0167 | 9.6 | 11.67 |
| 79. | III | 10.0 | PO₄³⁻ | 1.586 | 0.167 | 0.96 | 11.73 |
| 80. | III | 0.1 | HPO₄²− | 0.001586 | 0.000167 | 9.6 | 7.65 |
| 81. | III | 0.1 | HPO₄²− | 0.01586 | 0.00167 | 0.96 | 7.80 |
| 82. | III | 0.1 | HPO₄²− | 0.1586 | 0.0167 | 0.096 | 8.04 |
| 83. | III | 1.0 | HPO₄²− | 0.001586 | 0.000167 | 96 | 7.68 |
| 84. | III | 1.0 | HPO₄²− | 0.01586 | 0.00167 | 9.6 | 7.84 |
| 85. | III | 1.0 | HPO₄²− | 0.1586 | 0.0167 | 0.96 | 7.96 |
| 86. | III | 10.0 | HPO₄²− | 0.01586 | 0.00167 | 96 | 8.01 |
| 87. | III | 10.0 | HPO₄²− | 0.1586 | 0.0167 | 9.6 | 8.06 |
| 88. | III | 10.0 | HPO₄²− | 1.586 | 0.167 | 0.96 | 8.19 |
| 89. | III | 0.1 | H₂PO₄⁻ | 0.001586 | 0.000167 | 9.6 | 7.54 |
| 90. | III | 0.1 | H₂PO₄⁻ | 0.01586 | 0.00167 | 0.96 | 7.36 |
| 91. | III | 0.1 | H₂PO₄⁻ | 0.1586 | 0.0167 | 0.096 | 6.48 |
| 92. | III | 1.0 | H₂PO₄⁻ | 0.001586 | 0.000167 | 96 | 7.72 |
| 93. | III | 1.0 | H₂PO₄⁻ | 0.01586 | 0.00167 | 9.6 | 7.42 |
| 94. | III | 1.0 | H₂PO₄⁻ | 0.1586 | 0.0167 | 0.96 | 6.58 |
| 95. | III | 10.0 | H₂PO₄⁻ | 0.01586 | 0.00167 | 96 | 7.53 |
| 96. | III | 10.0 | H₂PO₄⁻ | 0.1586 | 0.0167 | 9.6 | 6.50 |
| 97. | III | 10.0 | H₂PO₄⁻ | 1.586 | 0.167 | 0.96 | 6.14 |

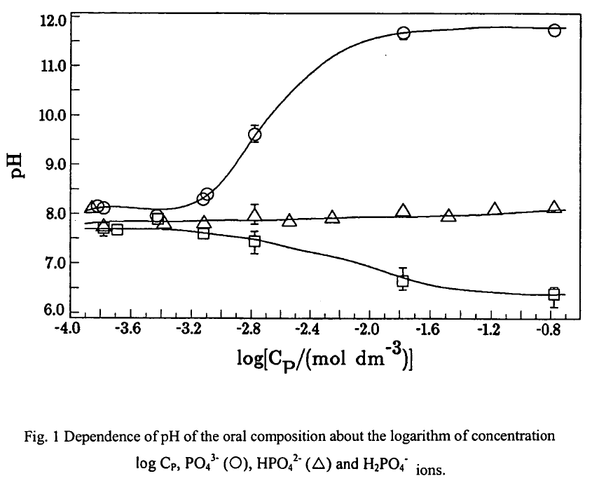

The results displayed in the Table 1 reveal that the pH of oral composition that contains phosphatic component in the form PO₄³⁻ is determined by the concentration of phosphatic ions regardless of the type of zeolite and his amount in the oral composition; pH of oral composition does not change significantly with the increase of concentrations of PO₄³⁻ ions in the area of concentration from about 0,00015 mol dm⁻³ to about mol 0,0008 mol dm⁻³ (state of equilibrium of pH is from about 8.12 to about =8.5). (See FIG. 1). At concentrations PO₄³⁻ of ions greater than 0,0008 mol 3 pH grows with the increase of the concentration of dm⁻³ ions and reaches maximal pH from about 11.7 for concentrations of PO₄³⁻ ions that are greater than 0,0167 mol dm⁻³.
pH of oral composition that contains phosphatic component in the form of HPO₄⁻ ions changes very little with the change of concentration of HPO₄⁻ ions; pH does change from about 7.80 to 8.11 when the concentration of HPO₄′ ions changes (increase) increase three times in size, that is from 0.00014 mol dm⁻³ to 0,167 mol dm⁻³ (see FIG. 1).
Finally, the pH of oral composition that contains phosphatic component in the form of H₂PO₄⁻ ions, is for low concentration of H₂PO⁴⁻ ions (of from about 0.00017 mol dm⁻³ to about 0,0008 mol dm⁻³) constantly (app. 7.7), progressively decreases with increased concentration of H₂PO₄⁻ greater than 0,0008 mol dm⁻³ and reaches pH value from about 6.4 at concentrations of H₂PO₄⁻ ions from about 0.17 mol dm⁻³.

[image]

The results displayed in the Table 1 and FIG. 1 reveal that by the controlled use of calcium form of zeolite in the range of 0.1 wt. % to 10 wt. % and phosphate ions in the range of 0 wt. % to 1,586 weight %, (what corresponds to molar concentrations of phosphate ions in the range of 0 mol dm⁻³ to 0.167 mol dm⁻³), molar ratio Ca/P in the oral composition for the stabilization, (re)calcification and (re)mineralization of dental enamel and dentine can be adjusted in the range of Ca/P=0.096 to Ca/P=178 (Ca/P=∞ in the absence of phosphates) and that the pH of the oral composition for the stabilization, (re)calcification and (re)mineralization of dental enamel and dentine can be controllably adjusted in the range of the pH=4.88 to pH=11.82. One must stress that the type of zeolite, its quantity and dilution of oral composition does not considerably affect the given pH value characteristic for the given concentration of phosphate ions.

The results displayed in the Table 1 and FIG. 1, also lead to the conclusion that the hydrogen phosphate (HPO₄²⁻) is the best form of phosphate ions for regulation of pH in the mouth cavity, in the optimal area.

The influence of pH on the process of demineralization, and thus to the stability of teeth has been tested on the sample of 15 teeth. For this purpose, each sample of teeth is divided into two parts. One part of the sample of every teeth is treated for 9 minutes in suspension which contained 1 g of calcium form of zeolite of the type I in 10 (5 teeth) and 100 ml of deminaralised water (5 teeth), or of the type II in the 10 ml of deminaralised water (5 teeth) (pH=8.02-9.05), respectively. Following the treatment, teeth are washed with deminaralised water, and dried. Dried treated and untreated teeth were deminaralised for 12 hours in the solution of acetic acid 4 M (pH=3.5) at temperature of 37° C. in dynamic conditions (mixing the suspension with samples of teeth).

Following the finished demineralization, the concentration of calcium ions in the group of controlled, untreated teeth, generally was around 0.42 and 0.5 mg/ml while the concentration of calcium ions in the groups of teeth treated with the preparation, was statistical reduced after the demineralization for app. 10% (see FIG. 2).

[image]

Obtained result is explained with remineralization (recalcification) of dental enamel with the synchronous stabilization during the treatment in the alkaline suspension of calcium form of zeolite and accordingly, slower process of demineralization in the acid medium (0.4 M acetic acid; pH=3.5).

In order to prove the conclusion which resulted from the testing of impact of pH on the stability of teeth, samples of teeth were treated with the solutions made in the following manner: (i) by extraction of solid stage (calcium form of zeolite) from the suspension (1 g of calcium form of zeolite in the 100 ml deminaralised water; pH=8.01) 24 hour following the preparation with the continuously mixing (solution (L-Ca)₀), and (ii) by extraction of solid stage (sodium form of zeolite of the type I; pH=10.5) from the suspension 24 hour following its preparation, with the continuous mixing (solution (L-Na)₀). The solution (L-Ca)₀ contained 0.01 mg Ca²⁺ ions/cm³ (see Table 3) as a consequence of equilibrium of the process of substitution H+ ions from the water according to the equation:
CaZ+2H₂O<=>H₂Z+Ca²⁺+2OH⁻ (1)

Each of the sample tooth (4 for treatment with solution (L-Ca)₀ and 3 for the treatment with solution (L-NA)₀ was divided in two parts. One part of teeth, treatment with solution (L-Ca)₀ or (L-Na)₀ (12 hours at 37° C. with mixing), and second part of the teeth was treated under the same conditions with 0.4 M solution of acetic acid (solution K). Following the treatment, in the solutions was ascertained the concentration of Ca²⁺ ions, with the AAS method. The results are displayed in the Table 2 as concentrations of Ca²⁺ ions in the solutions (L-Ca)₁-(L-Ca)₄ obtained after the treatment of samples of teeth 14 with the solution (L-Ca)₀, (L-Na)₅-(L-Na)₇ obtained after treatment of samples of teeth 5-7 with the solution (L-Na)₀ and K₁-K₇, obtained following the treatment of samples of teeth 1-7 with 0.4 M solution of acetic acid (solution K).

TABLE 2
The influence of the manner of treatment of teeth on their stabilization

| | CONCENTRATION |
|---|---|
| SAMPLE | OF Ca²⁺ (mg/cm³) |
| (L-Ca)₀ | 0.01 |
| (L-Na)₀ | 0.0013 |
| (L-Ca)₁ | 0.0017 |
| K₁ | 3.446 |
| (L-Ca)₂ | 0.0022 |
| K₂ | 4.052 |
| (L-Ca)₃ | 0.0029 |
| K₃ | 2.430 |
| (L-Ca)₄ | 0.0036 |
| K₄ | 3.84 |
| (L-Na)₅ | 0.0012 |
| K₅ | 3.187 |
| (L-Na)₆ | 0.00029 |
| K₆ | 3.516 |
| (L-Na)₇ | 0.00092 |
| K₇ | 1.995 |

The results in the Table 2 display that the concentration of calcium in the alkali solutions (L-Ca)j-(L-Ca)₄ are app. 1440 times smaller than in the acid solutions K₁-K₄, and that the concentration of calcium in the acid solutions (L-Na)₁-(L-Na)₃ is app. 5600 times smaller than in the acid solutions K5-K7, after the contact with the samples of teeth.

This means that the decalcification in the alkali solutions has being reduced for more than 3 three orders of the value (pH=8.01) or more (pH=10.5) in respect to decalcification in the acid solutions (pH=3.5). Although the greater effect of stabilization at greater pH value (solution (L-Na)₀; pH=10.5) is expected, the reduction of concentration of calcium in the solution (L-Ca)₀ of 0.01 mg/cm³ on app. 0,0026 mg/dm³ clearly shows that during the contact of the sample of teeth with the solution (L-Ca)₀ app. 75% of calcium ions from the solution were <<bonded>> on the dental enamel. In other words, alkali medium established by the presence of calcium form of zeolite not only significantly slows down the process of decalcification, but stimulates the process of calcification.

In order to prove the process of (re)calcification during the treatment and to discern the influence of stabilization of dental enamel in the alkali environment from the impact of (re)calcification on the reduction of demineralization in the sour medium, the samples of teeth have been divided into two parts and after that the samples were treated as follows:

Series A (3 samples): one half of each three teeth from the series A is deminaralised for 6 hours in the sour medium in the before mentioned manner (exposure of teeth to 0.4 M solution of acetic acid at temperature of 37° C. in the dynamic conditions), and the second half of each three teeth from the series A is, before the demineralization, treated during 60 min with the suspension which contained 1 g of calcium form of zeolite of the type I in 100 ml of deminaralised water.

Series B (3 samples): one half of each three teeth from the series B is deminaralised for 6 hours in the sour medium in the same way as the samples from the series A, and the second half of each three teeth from the series B is, before the demineralization, treated during 60 min with the suspension which contained 1 g of calcium form of zeolite of the type I in 100 ml of deminaralised water.

TABLE 3
The influence of inserting of calcium ions in
the enamel on the stabilization of teeth

| | Concentration | | |
|---|---|---|---|
| Sample No.: | of Ca²⁺ (mg/ml) | X+ (%) | 100 − X+ (%) |
| A1 | 0.744 | — | — |
| A1-(Ca) | 0.582 | 75 | 25 |
| A2 | 0.642 | — | — |
| A2-(Ca) | 0.556 | 86.6 | 13.4 |
| A3 | 0.632 | — | — |
| A3-(Ca) | 0.506 | 80 | 20 |
| BI | 0.788 | — | — |
| BI-(Na) | 0.582 | 88 | 12 |
| B2 | 0.655 | — | — |
| B2-(Na) | 0.595 | 91 | 9 |
| B3 | 0.620 | — | — |
| B3-(Na) | 0.631 | 101.8 | −1.8 |

The concentrations of calcium in the solutions after the finished decalcification in the sour medium are shown in the table 3. The marks A1-A3 correspond to the solutions after the decalcification of teeth from the series A, marks B1-B3 correspond to the solutions after the decalcification of teeth from the series B, marks A1-(Ca)-A3-(Ca) correspond to the solutions after the decalcification of teeth from the series A, treated with the suspension which contained 1 g of calcium in form of zeolite of the type I in 100 ml of deminaralised water and mark B 1-(Na)-B3-(Na) correspond to the solution which contained 1 g of sodium form of zeolite of the type I in 100 ml of deminaralised water. As was expected, the concentrations of calcium in the solutions A-(Ca) and B-(Na) are 9-25% lower than in the solutions A and B. However, the average reduction of concentration of calcium in the solutions A-(Ca) amounts to 19.5%, while the average reduction of concentration of calcium in the solutions B-(Na) is 6.4%. Hence one can conclude that the increased level of stabilization of teeth in the suspension of calcium forms of zeolite in comparison with the suspension of sodium form of zeolite is caused by the incorporation of calcium from the calcium form of zeolite into the dental enamel (calcification) during the treatment.

With the purpose to determination of the assumption about the concurrent incorporation of calcium and phosphate ions into the dental enamel and dentin and the creation of hydroxyapatite (remineralization), the samples of teeth were treated with the solution which contained calcium and phosphate ions. The solution is prepared by extraction of clear liquid stage from the freshly prepared oral composition which contained 1 g calcium form of zeolite of the type I in the 100 ml 5×10⁻⁴ M of solution Na₂HPO4. Thus prepared solution possessed pH 7.8 and contained 3.1×10⁴ mol dm⁻³ phosphate ions and 1.2×10⁻⁴ mol dm⁻³ of calcium ions. The solution is divided into 5 equal aliquots of 10 ml each, and in the each aliquot (10 ml of solution mixed with magnetic mixer) is put one tooth of approximately the same mass. The moment of placing the teeth into the solution is marked as zero time (t=0) of the process of

[image]

remineralization. In certain times, t, following the beginning of the process of remineralization, aliquot samples were taken from 1 ml solution in which are found the concentrations of calcium and phosphate. The quantities of calcium and phosphate ions, expressed as a quantity of hydroxyapatite incorporated in the tooth (see FIG. 3), have been calculated from the difference between the initial concentrations of calcium and phosphate ions in the solution and the concentrations of the same ions after certain time of remineralization. The results are displayed in the FIG. 3, as an arithmetic mean of the quantities of hydroxyapatite laid down on the tooth at different times of the process of remineralization. The quantity of hydroxyapatite laid down on the tooth is linear function of time of remineralization, which is in the above mentioned example completed in app. 10 min.

It is important to emphasize that the speed of remineralization and the quantity of deposited hydroxyapatite can be adjusted with the concentrations of phosphate and calcium form of zeolite in the suspension. It is especially significant that the redundancy of one of the components (calcium and phosphate ions) does not modify the chemical composition of deposited hydroxyapatite, i.e. that the quantity of deposited hydroxyapatite is determined by the concentration of component that is lacking (in the above mentioned case, by the concentration of calcium ions).

Based on the presented results, it was determined that the reduction of calcium concentrations in acid solutions, after the treatment of tooth samples with oral composition in respect to the untreated samples, one can ascribe the stabilization of mineral portion of tooth (enamel and dentine) to the activity of excess of OH⁻ ions in the mildly alkaline environments and concurrently by incorporating calcium and phosphorus from the oral composition into the enamel and dentine (remineralization). Since the demineralization is a time dependent process and that the demineralization and (re)remineralization are parallel processes, one can presume that the time of treatment of teeth with oral composition suspension can greatly affect the stabilization, (re)calcification and (re)mineralization of teeth.

The results of the testing of the time influence in the treatment of teeth with oral composition, according to the invention, are proven to depend on the cumulative time of the treatment (10-300 min), the stability of dental enamel and dentine increases from 7 to 29% in respect to untreated teeth (see FIG. 4).

[image]

Results displayed in FIG. 4, undoubtedly reveal that the value S % increases with the time spent on treatment; S % has it's maximum at S≈23% in app. 60 min, and any further treatment does not increase it significantly. Therefore one can conclude that the reduction of demineralization after the treatment with oral composition caused by stabilization of dental enamel, by control of pH amount with the synchronous (re)calcification, (re)mineralization, by release of active calcium from the zeolite and to its incorporation into the enamel, together with equivalent quantity of phosphate, with the help of matrix proteins, under the optimal pH. It was unexpectedly shown that with the addition of matrix protein, the efficiency of the composition has been improved, as follows: the increase of the rate of stabilization of dental enamel i.e. the time needed for achieving the full effect of stabilization has being reduced. Measured mean value of the preparation with the matrix protein has shown the improvement of 30 to 50% in regard to composition without the matrix protein.

The best effect in the application of oral composition is achieved, according to the invention, when components of oral composition are physically separated. Above mentioned physical separation can be implemented: (a) using the chambers separated by impermeable barrier in the tube (toothpaste), (b) micro encapsulation of one of the components (toothpaste, chewing gum, bonbons, gel) or (c) mixing the components of oral composition into different layers (chewing gum, bonbon).

Realization of the Innovation

Oral composition according to the invention can be used in the form of dental paste, chewing gum, gel, water for rinsing the mouth, bonbons and others preparations that stay in the mouth cavity.

In the event the invention is available in the form of dental paste, the components, according to the invention, are present in the following quantities:
1. Calcium form of zeolite: 0.1-10 wt. %
2. Phosphate ions: 0.00132-2 wt. %
3. Matrix proteins: 1-5 wt. % in relation to the phosphate ions

Except the composition according to the invention, the toothpaste can incorporate:
Abrasive (silica dioxide in different forms, aluminum hydroxide, aluminum oxide or the mixture of the same).
Thickening agents (glycerine, propylene glycol, polyethylene glycol, mannitol, sorbite, mineral oils, vegetable oils or the mixture of the same).
Bonding agents (synthetic polymers soluble in water, agar-agar, pectins, carboxylmethyl cellulose, xanthic rubber, carboxyl vinyl polymers, polyvinyl alcohol, polyvinyl pyroles, carogene, trogakant rubber, guar, cellulose, methyl hydrosipropyll cellulose or the mixture of the same).
Surface active substances (Na-N-laurylsarkozinate, Na-lauryl sulphate, palm oil, the coconut oil or the mixture of the same).
Sweeteners (Na-saccharinate, Na-cyklamate, sorbitol, xyilitol, lactosis, maltose, fructose or the mixture of the same).
Corigenses of taste (the mint oil, spearmint oil, chamomile oil, sage oil, eucalyptus oil, the oil of tea plant, thyme oil, cinnamon oil, fennel oil, cardamom oil or the mixture of the same).
Solvents (lesser polyhydroxylated alcohols and ethers or mixtures of the same).

The given examples of abrasive, thickening agents, bonding agents, surface acting agents, sweeteners, corigenses of taste and solvents in no way limit all possible substances that can be used for same purpose.

In the event the invention is designed in the form of chewing gum, according to the invention, the next components are present in the following quantities:
1. Calcium form of zeolite: 0.1-10 wt. %
2. Phosphate ions: 0.00132-2 wt. %
3. Matrix proteins: 1-5 wt. % in relation to the phosphate ions
4. standard gum-bases, standard plasticizers, standard sweeteners, standard eastemers, standard filling agents, standard softeners, standard emulsifying agents, standard colors and standard aromas.

In this patent application are presented some specific implementations of this innovation. The professionals in this area know that there are various versions of this innovation possible. One must emphasize that all such kinds of realizations and implementations of this innovation are included within the patent applications which follow.

1. An oral composition for the stabilization, recalcification and remineralization of dental enamel and dentine and adjustment of the pH of the oral cavity to a range of from about 7.5 to about 11.9 comprising calcium forms of zeolite, a source of phosphate ions and matrix proteins.

2. The composition of claim 1, where the molar ratio between calcium and phosphate, Ca/P, is from about 0.096 to about 178.

3. The composition of claim 1 further comprising at least one compound selected from abrasives, thickening agents, surface acting agents, sweeteners, taste corigenses and solvents.

4. The composition of claim 3, characterized by that it controls and adjusts pH of the mouth cavity in range from about 8.12 to about 8.50.

5. The composition of claim 1, comprising from about 0.1 to about 10 weight percent of calcium form of zeolite and from about 0.00132 to about 1.586 weight percent of phosphate ions.

6. The composition of claim 1, where the concentration of the phosphate ions in the preparation amounts in interval from about 0.000129 mol dm⁻³ to about 0.167 mol dm⁻³.

7. The composition of claim 1, where the weight proportion of matrix proteins in the composition is in range from about 1 wt. % to about 5 wt. % in relation to the phosphate ions.

8. The composition of claim 1 further comprising extract and/or oil of the medicinal herb milfoil in the amount from about 0.01 wt. % to about 0.025 wt. %.

9. The composition of claim 1, comprising 0.1-10 wt. % of calcium form of zeolite, 0.00132-2 wt. % of phosphate ions and 1-5 wt. % of matrix proteins in relation to the phosphate ions, and the remainder of the composition comprises abrasives and/or thickening agent and/or binding agents and/or surface acting agents and/or sweetener and/or corigenses of taste and/or solvents.

10. The composition of claim 1 in the form of the chewing gum.

11. The composition of claim 1 in the form of bonbons.

12. The composition of claim 1 in the form of tooth paste.

13. The composition of claim 1 in the form of fluid for rinsing the mouth cavity.

14. The composition of claim 1 where the components are physically separated.

15. The composition of claim 10 where the oral composition components are located in the different layers.

16. The composition of claim 9 where the oral composition components are microencapsulated.

17. A method of remineralizing and recalicifying dental enamell and dentine, comprising administering to a patient in need thereof, a remineralizing and recalicifying amount of the composition of claim 1.

18. The method of claim 17 wherein the patient is selected from the group consisting of pregnant women, older persons and children.
